(19) United States
(12) Patent Application Publication
(10) Pub. No.: US 2013/0330322 A1
(43) Pub. Date: Dec. 12, 2013

(54) Methods of Treating Skin Aging With Toll-Like Receptor 7 Agonists

(71) Applicant: Metabeauty, Inc., Miami Beach, FL (US)

(72) Inventor: Leslie Baumann, Miami Beach, FL (US)

(73) Assignee: Metabeauty, Inc., Miami Beach, FL (US)

(21) Appl. No.: 13/967,064

(22) Filed: Aug. 14, 2013

Related U.S. Application Data
(60) Provisional application No. 61/682,767, filed on Aug. 14, 2012.

(51) Int. Cl. A61K 8/49; A61Q 19/00; A61K 8/67; A61Q 19/08

(52) U.S. Cl. CPC A61K 8/4953; A61K 8/49; A61K 8/4946; A61Q 19/08; A61Q 19/007; A61Q 19/00; A61K 8/671
USPC 424/94.67; 514/260.1; 514/263.37; 514/293; 514/265.1; 514/263.3

(57) ABSTRACT

A method of reducing the appearance of skin aging comprising the step of applying a topical composition to the skin of a human exhibiting one or more signs of skin aging selected from fine lines, wrinkles, decreased skin elasticity, decreased light reflection, uneven pigmentation (dyschromia), coarse texture, dryness, solar lentigos, seborrheic keratoses, decreased dermal thickness, and/or increased sebum production, wherein the composition consists essentially of a dermatologically acceptable carrier and a targeted immune response modifier selected the group consisting of: (i) a 5-amino thiazolo[4,5-d]pyrimidin-one compound; (ii) an 8-hydroxyadenine compound; (iii) an imidazo-1,5-napthyridine compound; (iv) a 2-substituted-8-oxodeazapurine; (v) methyl 2-(3-{[6-amino-2-butoxy-8-oxo-7H-purin-9(8H)-yl]methyl}phenyl)acetate; (vi) 2-(3-{[6-amino-2-butoxy-8-oxo-7H-purin-9(8H)-yl]methyl}phenyl)acetic acid; and (iii) methyl 2-(3-(((3-(6-amino-2-butoxy-8-oxo-7H-purin-9(8H)-yl)propyl)(3-(dimethylamino)propyl)amino) phenyl)acetate and (vii) 2-(3-(((3-(6-amino-2-butoxy-8-oxo-7H-purin-9(8H)-yl)propyl)(3-(dimethylamino)propyl)amino)phenyl)acetic acid.

### CROSS REFERENCE TO RELATED APPLICATIONS

This application claims priority to U.S. Provisional Application Ser. No. 61/682,767 filed on Aug. 14, 2012, the disclosure of which is incorporated herein by reference in its entirety.

### FIELD OF THE INVENTION

The present invention relates to a method of improving the appearance of aging skin by applying a topical composition that includes a targeted toll-like receptor 7 agonist in a dermatologically acceptable carrier.

The present invention is further directed to topical application of the inventive compositions in a safe and effective amount such that expression of genes that code for Interferon α subtypes including IFNA1, IFNA2, IFNA5, IFNA6, and IFNA8, MyD88, Collagen Types 1 and/or 3 (COL1 and/or COL3) and/or Tissue Inhibitor of Matrix Metalloproteinases (TIMP1) are increased and genes that code for matrix metalloproteinases, notably MMP-1, MMP-2 and/or MMP-9 are downregulated. (Levels of gene expression are determined according biological assays well-known in the art and exemplified in the procedures described hereinbelow.)

### STATEMENT OF FEDERALLY SPONSORED RESEARCH OR DEVELOPMENT

Not applicable.

### BACKGROUND OF THE INVENTION

U.S. Pat. No. 7,521,459 (Baumann et al.) teaches a method of inducing an immune cytotoxic response in a section of normal photodamaged dermal or epidermal tissue of a patient exhibiting fine lines and clinical wrinkles, where the section of tissue is not being treated for viral infection or skin cancer. The disclosed method involves topically applying an effective amount of a cosmetically or dermatologically acceptable composition comprising an immunomodulatory compound—in particular, 1-isobutyl-1H-imidazo[4,5,-C]quinolin-4-amine (Imiquimod sold as Aldara®)—capable of attracting macrophage cells to the area surrounding the section of tissue exhibiting fine lines and clinical wrinkles. An improved appearance (i.e., reduction in the appearance of fine lines and wrinkles) is taught to be visible within at least 4 weeks of application of the immunomodulatory compound.

### SUMMARY OF THE INVENTION

A method of reducing the appearance of skin aging comprising the step of applying a topical composition to the skin of a human exhibiting one or more signs of skin aging selected from fine lines, wrinkles, decreased skin elasticity, decreased light reflection, uneven pigmentation (dyschromia), coarse texture, dryness, solar lentigos, seborrheic keratoses, decreased dermal thickness, and/or increased sebum production wherein the composition consists essentially of a dermatologically acceptable carrier and a targeted immune response modifier selected from the group consisting of: (i) a 5-amino thiazolo[4,5-d]pyrimidin-one compound; (ii) an 8-hydroxyadenine compound; (iii) an imidazo-1,5-napthyridine compound; (iv) a 2-substituted-8-oxodeazapurine; (v) methyl 2-(3-{[6-amino-2-butoxy-8-oxo-7H-purin-9(8H)-yl]methyl}phenyl)acetate; (vi) 2-(3-{[6-amino-2-butoxy-8-oxo-7H-purin-9(8H)-yl]methyl}phenyl)acetic acid; and (iii) methyl 2-(3-(((3-(6-amino-2-butoxy-8-oxo-7H-purin-9(8H)-yl)propyl)(3-(dimethylamino)propyl)amino)phenyl)acetate and (vii) 2-(3-(((3-(6-amino-2-butoxy-8-oxo-7H-purin-9(8H)-yl)propyl)(3-(dimethylamino)propyl)amino)phenyl)acetic acid.

### DETAILED DESCRIPTION OF THE INVENTION

The present invention is directed to methods of treating and/or preventing skin aging, caused both extrinsically and intrinsically, and characterized by clinically observed and/or instrumentally measured changes in one or more of (a) number and depth of fine lines and/or wrinkles, (b) skin elasticity, (c) radiance (light reflection), (d) uneven pigmentation (dyschromia), (e) smoothness (texture), (e) dryness (moisture content), (f) solar lentigos, (g) seborrheic keratoses (h) dermal thickness and/or (i) sebum production. Collectively, these methods are referred to hereinbelow as methods of reducing the appearance of skin aging.

In a first embodiment, the present invention is directed to a method of reducing the appearance of skin aging that includes the step of applying to an area of skin presenting with the appearance of skin aging a topical composition that consists essentially of an immune response modifier that is a 5-amino thiazolo[4,5-d]pyrimidin-2-one compound in a dermatologically acceptable carrier.

Preferred 5-amino thiazolo[4,5-d]pyrimidin-one compounds for inclusion in the topical composition useful in the practice of the first embodiment of the method of the present invention are 5-amino-3-((2R,3R,4S,5R)-3,4-dihydroxy-5-(hydroxymethyl)tetrahydrofuran-2-yl)thiazolo[4,5-d]pyrimidine-2,7(3H,6H)-dione (isatoribine, structure I), and its acetylated analog and prodrug, 5-amino-3-((2R,3R,4S,5R)-3,4-diacetyl-5-(hydroxymethyl)tetrahydrofuran-2-yl)thiazolo[4,5-d]pyrimidine-2(3H,6H)-one (ANA-975, structure II).

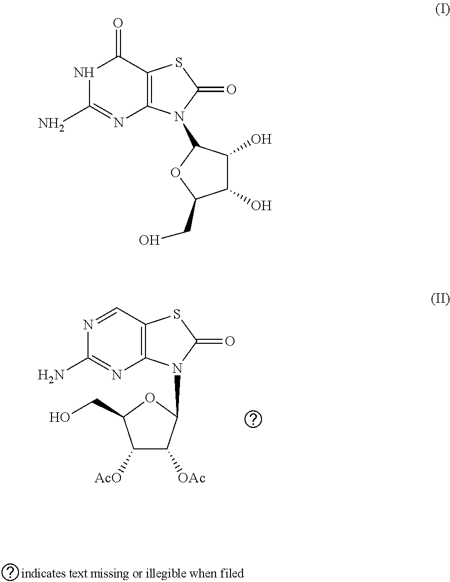

The compound of structure II is a prodrug of the compound of structure I.

The synthesis of 5-amino-3-((2R,3R,4S,5R)-3,4-dihydroxy-5-(hydroxymethyl)tetrahydrofuran-2-yl)thiazolo[4,5-d]pyrimidine-2,7(3H,6H)-dione is described in U.S. Pat. No. 5,041,426 (Robins et. al.). The synthesis of 5-amino-3-((2R,3R,4S,5R)-3,4-diacetyl-5-(hydroxymethyl)tetrahydrofuran-2-yl)thiazolo[4,5-d]pyrimidine-2(3H,6H)-one (ANA-975) is described in Alan X. Xiang et al., Discovery of ANA975: An Oral Prodrug of the TLT-7 Agonist Isatoribine, 26 Nucleosides, Nucleotides, and Nucleic Acids, 635-640 (2007).

Further examples of 5-amino-3-(tetrahydrofuran-2-yl)thiazolo[4,5-d]pyrimidine-2(3H,6H)-one compounds having a furanosyl (especially arabanosyl) substituent the 3N position that can be included in the topical composition used in the practice of this first embodiment of the present invention include compounds conform to structure III:

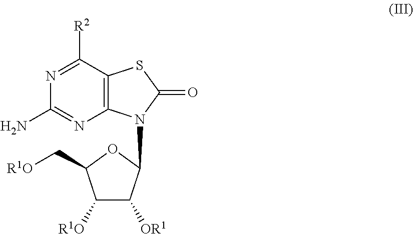

in which R¹ is independently H, —C(O)R₃, or a racemic, L-, or D-amino acid group —C(O)CHNH₂R⁴, wherein R³ is a substituted or unsubstituted alkyl, and R⁴ is H, or a substituted or unsubstituted alkyl; R² is H, OR⁵, or N(R⁶)₂ wherein R⁶ is independently H or alkyl, and wherein R⁶ is independently H, substituted or unsubstituted alkyl, cycloalkyl, or together with nitrogen forms a substituted or unsubstituted heterocycloalkyl ring; and wherein if R² is —OH, at least one of the R¹ groups is a racemic, L-, or D-amino acid group —C(O)CHNH₂R⁴. Such 5-amino-3-(tetrahydrofuran-2-yl)thiazolo[4,5-d]pyrimidine-2(3H,6H)-one compounds are described, for example, in U.S. Pat. No. 6,924,271 (Averett et al.).

In a second embodiment, the present invention is directed to a method of reducing the appearance of aging that includes the step of applying to an area of skin presenting with the appearance of skin aging a topical composition that consists essentially of an immune response modifier that is an 8-hydroxyadenine compound in a dermatologically acceptable carrier.

Preferred 8-hydroxyadenine compounds for inclusion in the topical compositions useful in the practice of this second embodiment of the method of present invention include 9-(3-carboxymethylbenzyl)-2-butoxy-8-hydroxyadenine [i.e. 2-(3-{[6-amino-2-butoxy-8-oxo-7H-purin-9(8H)-yl]methyl}phenyl)acetic acid, SM-324406]; 9-(3-carboxymethylbenzyl)-2-butoxy-8-hydroxyadenine, methyl ester [i.e. methyl 2-(3-{[6-amino-2-butoxy-8-oxo-7H-purin-9(8H)-yl]methyl}phenyl)acetate, SM-324405]; 2-butoxy-9-((N-(3-dimethylaminopropyl)-N-(3-carboxymethylbenzyl)-3-aminopropyl)-8-hydroxyadenine [i.e. 2-(3-(((3-(6-amino-2-butoxy-8-oxo-7H-purin-9(8H)-yl)propyl)(3-(dimethylamino)propyl)amino)phenyl)acetic acid, AZ12443988]; and 2-butoxy-9-((N-(3-dimethylaminopropyl)-N-(3-carboxymethylbenzyl)-3-aminopropyl)-8-hydroxyadenine, methyl ester [i.e. methyl 2-(3-(((3-(6-amino-2-butoxy-8-oxo-7H-purin-9(8H)-yl)propyl)(3-(dimethylamino)propyl)amino)phenyl)acetate, AZ12441970].

Other 8-hydroxyadenine compounds (aka 8-oxyadenine compounds) useful in the practice of the second embodiment of the present invention conform to the structure IV (shown in tautomeric form),

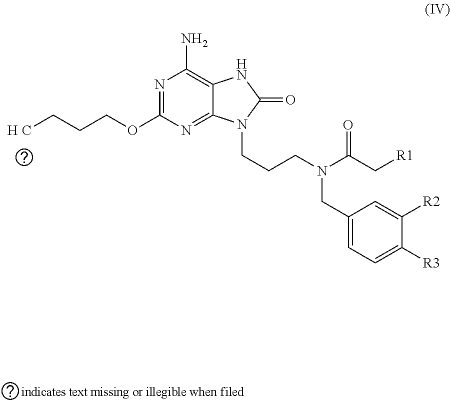

in which R1 is selected from CH₂CH₂N(Me)₂, 4-(HOCH₂CH₂)-1-Piz, and t-BuOCOCH₂; and R2 and R3 are selected from H and CH₂CO₂Me.

The synthesis of the 8-hydroxyadenine compounds useful in the practice of the present is described in International Patent Application Publication WO 2008/004948 (Cook et al.).

Still further 8-hydroxyadenine compounds that can be included in the topical composition useful in the practice of the second embodiment of the present invention include the compounds of structures V(i), V(j), and V(k):

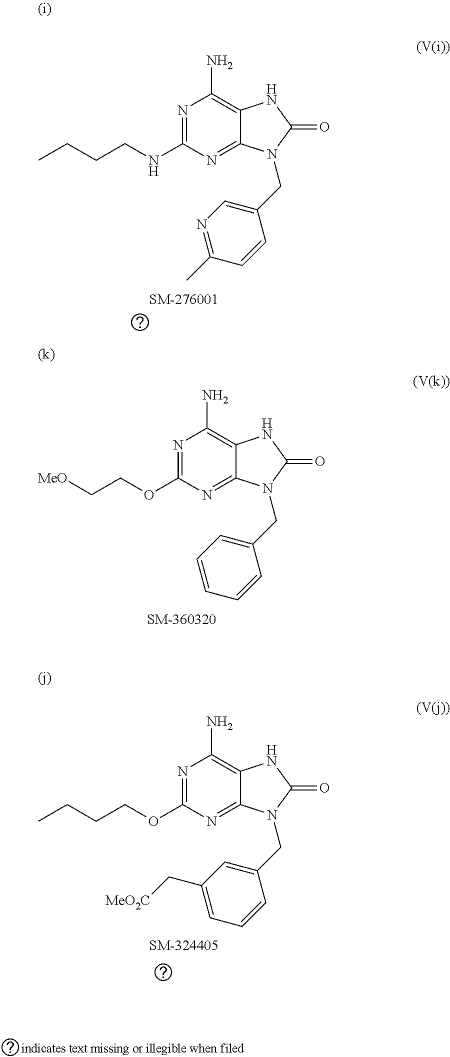 [SMILES: CCCCNc1nc(N)c2[nH]c(=O)n(Cc3ccc(C)nc3)c2n1 ; COCCOc1nc(N)c2[nH]c(=O)n(Cc3ccccc3)c2n1 ; CCCCOc1nc(N)c2[nH]c(=O)n(Cc3cccc(CC(=O)OC)c3)c2n1]

Still other 8-hydroxyadenine compounds that can be included in the topical compositions useful in the practice of this embodiment of the present invention are described in Kurimoto et al., Synthesis and Biological Evaluation of 8-Oxoadenine Derivatives as Toll-like Receptor 7 Agonists Introducing the Antedrug Concept, 53 J. Med. Chem. 22964-2972 (2010), incorporated herein by reference.

In a third embodiment, the present invention is directed to a method of reducing the appearance of aging that includes the step of applying to an area of skin presenting with the appearance of skin aging a topical composition that consists essentially of an immune response modifier that is an imidazo-1,5-napthyridine compound in a dermatologically acceptable carrier.

Preferred imidazo-1,5-napthyridine compounds to be included in the topical composition for use in the practice of the present invention are 2-methyl-1-(2-methylpropyl)-1H-imidazo[4,5-c][1,5]naphthyridin-4-amine; 1-(2-methylpropyl)-1H-imidazo[4,5-c][1,5]napthyridin-4-amine; and the compound of structure VI:

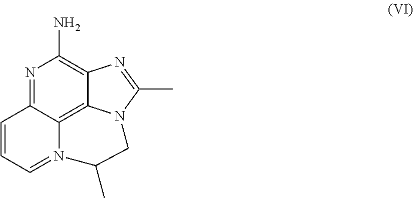

In yet a fourth embodiment, the present invention is directed to a method of reducing the appearance of skin aging that includes the step of applying to an area of skin presenting with the appearance of aging a topical composition that consists essentially of an immune response modifier that is a 2-substituted-8-oxodeazapurine of structure VII:

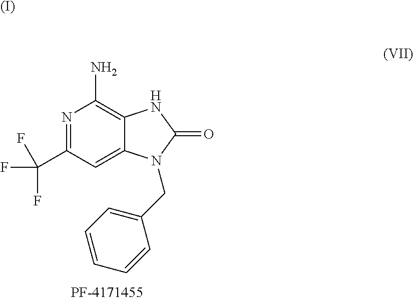 [SMILES: "(I)" ; Nc1nc(C(F)(F)F)cc2c1[nH]c(=O)n2Cc1ccccc1]

In a fifth embodiment, the present invention is directed to a method of reducing the appearance of skin aging that includes the step of applying to an area of skin presenting with the appearance of aging a topical composition that consists essentially of an immune response modifier that is selected from the group consisting of: (i) methyl 2-(3-{[6-amino-2-butoxy-8-oxo-7H-purin-9(8H)-yl]methyl}phenyl)acetate; (ii) 2-(3-{[6-amino-2-butoxy-8-oxo-7H-purin-9(8H)-yl]methyl}phenyl)acetic acid; and (iii) methyl 2-(3-(((3-(6-amino-2-butoxy-8-oxo-7H-purin-9(8H)-yl)propyl)(3-(dimethylamino)propyl)amino)phenyl)acetate and (iii) 2-(3-(((3-(6-amino-2-butoxy-8-oxo-7H-purin-9(8H)-yl)propyl)(3-(dimethylamino)propyl)amino)phenyl)acetic acid. The above compounds correspond to Structures VIII and IX below.

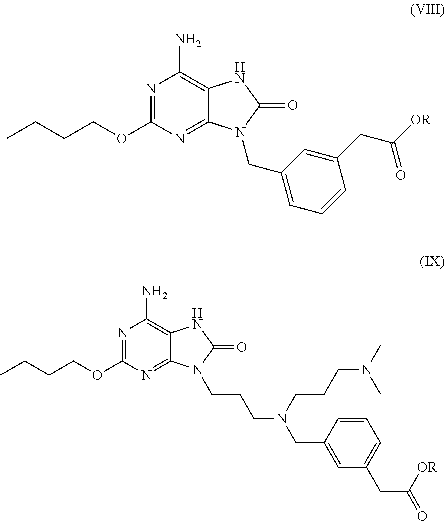

In embodiments where the immune response modifier corresponds to Structure VIII or IX, the R substituent can be methyl or hydrogen.

The topical compositions useful in the practice of all embodiments of the present invention include, as a required component, a dermatologically acceptable carrier. The composition may be a cream, lotion, gel, serum, spray or foam in the form of an oil-in-water emulsion, a water-in-oil emulsion, a water-in-oil-in-water emulsion, a water-in-silicone emulsion, a silicone-in-water emulsion, an aqueous dispersion or an anhydrous dispersion.

As used in the present application the phrase “dermatologically acceptable carrier” is to be understood by a person having ordinary skill as a composition whose ingredients alone and in combination are present at concentrations such that upon being placed into contact with non-cancerous human skin, the patient may experience transient irritation, but not sensitization or an allergic response or other adverse effects.

Important is that the dermatologically acceptable carrier not include any component that (i) would interfere with the ability of the topical composition, or its active component, to act as an agonist of toll like receptor 7 or (ii) would bind, sequester or otherwise inhibit the delivery of the active component to the skin exhibiting aging or (iii) would decrease the stability or desired shelf-life of the composition.

Preferably, the targeted immune response modifier useful in the practice of all embodiments of the present invention is present in a concentration of from about 0.001 percent to about 10 percent by weight of the total composition, more preferably at a concentration of from about 0.01 percent to about 5.0 percent, still more preferably at a concentration of from about 0.1 percent to about 1.0 percent.

The compositions of the present invention may be administered once or twice daily, as well as one or more times a week but less than once a day (e.g., once every other day, or once every third day, or twice a week application). Stronger doses of the compositions may be administered twice a month or monthly.

In certain embodiments, the compositions of the present invention may be administered once or twice monthly in combination with one of the following dermatologic/aesthetic procedures: (i) Light Emitting Diode (L.E.D.) Light Therapy; (ii) Intense Pulsed Light (I.P.L.) Therapy; (iii) laser skin resurfacing; (iv) mechanical exfoliation; (v) superficial, medium depth or deep chemical peels; (vi) radiofrequency treatment; (vii) ultrasound treatment; (viii) intradermal and intraepidermal injections with hyaluronic acid and derivatives thereof; (ix) cryosurgery or (x) botulinum toxin (applied topically or injected).

In still further aspects of the present invention, the topical composition according to one of the above embodiments further comprises at least one antioxidant and/or a retinoid.

Antioxidants (also referred to as anti-inflammatory agents) suitable for use in compositions of the present invention are well-known to those of ordinary skill in the art and include, but are not limited to: bioflavonoids (flavanols, flavonones, flavones, flavanolols, flavan-3-ols, isoflavones as described in K R Narayana, Indian J. Pharmacology, Vol. 33, pp. 2-16 (2001), the content of which is incorporated herein by reference); extracts of green tea; enzymes, including superoxide dismutase, catalase and glutathione peroxidase; vitamins and vitamin derivatives, including ascorbic acid, tocopherol, and derivatives thereof; ferulic acid; lycopene; carotenoids; Argan oil, resveratrol; idebenone, phloretin (3-(4-hydroxyphenyl)-1-(2,4,6-trihydroxyphenyl)propan-1-one) and Coenzyme-Q10. Additional antioxidant and anti-inflammatory agents, such as niacin and its derivatives (e.g., niacinamide), Cox-1 and Cox-2 inhibitors, steroids (e.g., hydrocortisone), fever few, aloe, licorice extract, chamomile, as well as those listed U.S. Pat. No. 6,492,326, respectively, at Column 9, line 53-Column 10, line 19 and Column 11, line 46-Column 13, line 12 (the disclosures of which are incorporated by reference in their entirety) are also suitable for use in the compositions of the present invention.

As used in the present application, the term “retinoid” is meant to include all natural and/or synthetic analogs of Vitamin A or retinol-like compounds which possess the biological activity of Vitamin A in the skin as well as the geometric isomers and stereoisomers of these compounds, such as all-trans retinoic acid and 13-cis-retinoic acid. Retinoids suitable for use in the present invention may be selected from the group consisting of retinol, retinal, retinol esters (C₂-C₂₂ alkyl esters of retinol, including retinyl palmitate, retinyl acetate, retinyl propionate), retinal, and/or retinoic acid (including all-trans retinoic acid and/or 13-cis-retinoic acid). Other retinoid suitable for use in the practice of the methods of the present invention include tocopheryl-retinoate [tocopherol ester of retinoic acid (trans- or cis-), adapalene {6-[3-(1-adamantyl)-4-methoxyphenyl]-2-naphthoic acid}, tazarotene (ethyl 6-[2-(4,4-dimethylthiochroman-6-yl)-ethynyl]nicotinate as well as the retinoids described in the following U.S. patents, the disclosures of which are incorporated herein by reference in their entirety: U.S. Pat. No. 4,677,120; U.S. Pat. No. 4,885,311; U.S. Pat. No. 5,049,584; U.S. Pat. No. 5,124,356.

Retinoids may be used at concentrations of from about 0.001% to about 10%, by weight of the topical composition in which they are formulated. Preferably retinoids are administered at a concentration of from about 0.001% to about 5% by weight of the composition, more preferably from about 0.01% to about 5%, most preferably from about 0.01% to about 3%.

Preferred retinoids are retinol, tretinoin (retinoic acid), adapalene {6-[3-(1-adamantyl)-4-methoxyphenyl]-2-naphthoic acid}, tazarotene (ethyl 6-[2-(4,4-dimethylthiochroman-6-yl)-ethynyl]nicotinate.

In a particularly preferred embodiment, a topical composition containing the immune response modifier according to the above-described embodiments of the present invention is applied in the morning and a topical composition containing the immune response modifier and a retinoid is separately applied in the evening.

The efficacy of the method of the several embodiments of the present invention in reducing the appearance of skin aging is demonstrated by the following non-limiting examples of clinical trials.

The following clinical studies are conducted to assess improvement in the appearance of aging skin after topical administration of compositions containing targeted immune response modifiers (hereafter “IRM”) described in the various embodiments according to the methods of the present invention.

Subjects demonstrating skin aging and having one of the following Baumann Skin Types, described below, are enrolled in a twelve-week, double blind, controlled study: (i) oily, sensitive, pigmented and wrinkled skin (OSPW); (ii) oily, resistant, pigmented and wrinkled skin (ORPW); (iii) dry, sensitive, pigmented and wrinkled skin (DSPW); (iv) dry, resistant, pigmented and wrinkled skin (DRPW).

OSPW and DSPW skin types are characterized by and predisposed to skin aging and either acne breakouts, facial redness and flushing, or frequent skin rashes and irritation. These inflammatory conditions are often followed by darkening of the skin in the areas of the pimples or skin irritation. Dark patches such as melasma and solar lentigos and seborrheic keratosis often occur.

ORPW and DRPW skin types rarely suffers from sensitivity such as acne breakouts, facial redness, or skin rashes; however, they are predisposed to skin aging and dark patches such as melasma, seborrheic keratosis and solar lentigos.

On Day 0, each enrolled subject is supplied with a package of individually coded tubes of cream, labeled “Left Arm”, “Right Arm”, and “Face”. Each tube contains one of the IRMs according to above-described embodiments at a concentration of 5% wt/wt in a dermatologically acceptable carrier or the carrier only (placebo). Labels are randomly assigned, with records separately maintained identifying the formulation. Codes are not known to either the subjects or the investigator until the study is completed and the data analyzed. All subjects receive Cetaphil cleanser, moisturizer and sunscreen for daily use throughout the study.

Study participants apply the cream to the arms and face once daily for 12 weeks. Clinical photographs are taken at weeks 2, 4, 8, and 12. In addition, subjects complete self assessments at each study visit. These assessments are validated using the Baumann Facial Aging Visual Scale, described in Appendix A.

Changes on Clinical Assessment; Instrumental Analysis and Histopathology

1—IRM Alone

Improvement in the appearance of fine lines and wrinkles is assessed by methods known in the art. Silflo replicas are taken at baseline and at week 12, with changes indicated by differences in the levels of shadows generated by incident lighting at the surface of Silflo replicas.

Reduction in facial superficial facial fine lines (SFLs) are also assessed using the method of Packman and Gans, “Topical Moisturizers: Quantification of their Effects on Superficial Facial Lines,” J. Soc. Cosmet. Chem. Vol. 29, pp. 79-90 (1978). At weeks 2, 4, 8 and 12, the number depth and shallowness of SFLs are scored within a defined area around the eye.

Skin firmness (elasticity) is assessed using a ballistometer, a device that drops a pendulum on the skin surface and measures the resultant bouncing pattern. More particularly, the ratio of the height of the first and second rebound peaks are calculated. Skin elasticity may also be measured using a Cutometer. Three measurements are taken and averaged to get the final Cutometer measurement.

Spectrophotometric Intracutaneous Analysis (SIAscopy) measures collagen based on optical scattering and absorption properties of the skin's chromophores (hemoglobin, melanin and collagen). After completion of the study, participants exhibiting pronounced SFLs show a statistically significant increase of collagen above baseline. This is also evidenced in an improved appearance of the skin, including increased elasticity.

Improved skin retention of water/skin moisture content is assessed via impedance measurements on the cheek area of subjects using a Novameter. Transepidermal water loss (TEWL) is measured using a Tewameter.

Sebum secretion is measured using a Sebometer and Sebutape.

After completion of the study, Silflo replicas show fewer fine lines in aging skin treated with IRMs according to methods of the present invention. In some participants, ballistometer and Cutometer measurements show increased skin elasticity. Some participants exhibit changes in SIAscopy showing collagen patterns representative of those seen in skin that does not exhibit signs of aging as discussed above. While SIAscopy can be considered less sensitive than gene expression tests (see below), it does provide a clinically validated non-invasive method to assess the skin's collagen content. Increase in skin collagen is also evidenced in an improved appearance of the skin (based on clinician graded photography and participant self assessment). Increased elasticity is observed in many participants as well as decreased skin fragility. Some patients report less bruising after mild trauma to the sites on their arm treated according the methods of the present invention. Improved skin retention of water/skin moisture content is seen in Novameter measurements and decreased transepidermal water loss is reflected in Tewameter measurements. Surprisingly, sebum secretion measurements are seen to increase only in the DSPW and DRPW skin types, but not in the OSPW and ORPW skin types.

Standard 2-4 mm skin punch biopsies are taken at pre-identified sites (on subject's arms) on Day 0 and at the Week 12 visit. Subjects apply the assigned formulation in the presence of the investigator and return for a biopsy within 4-6 hours.

Histopathological changes are observed after completion of the 12-week trial. In the epidermis, one or more of the following changes are noted in the skin at non-placebo study sites (i.e., site of application of study composition): normalization of keratinization with a basket weave stratum corneum; a more uniform epidermal thickness with a more ordered epidermal proliferation; a more regular rete ridge pattern (less flattening).

In the dermis, one or more of the following changes are noted in the skin at non-placebo study sites: increase in papillary dermal hyperplasia; increased dermal thickness; reduction in solar elastosis; a more uniform pattern of stromal cells within the papillary dermis; an increase of stromal cells within the area of solar elastosis; increased collagen staining; and/or increased hyaluronic acid staining.

2a—Combined Contemporaneous Treatment of Aging Skin with IRM and Retinoid

The following clinical studies are conducted to assess improvement in the appearance of aging skin after topical administration of compositions containing targeted immune response modifiers (hereafter “IRM”) in combination with a retinoid as described in the various embodiments according to the methods of the present invention.

Subjects demonstrating skin aging and having Baumann Skin Types ORPW and DRPW, as described above, are enrolled in a twelve-week, double blind, controlled study (Because OSPW and DSPW skin types are characterized by skin sensitivity, They were excluded from this study.)

On Day 0, each enrolled subject is supplied with a package of individually coded tubes of cream, labeled “Tube A” and “Tube B”. Tube A contains one of two formulations: 5% IRM+vehicle (carrier); or vehicle only (placebo); Tube B contains either 0.025% tretinoin in a cream formulation or vehicle only (placebo). The subjects apply Tube A to the arms and face, wait 5 minutes, and then apply the contents of Tube B over the same area. Labels are randomly assigned with records separately maintained identifying the formulation. Codes are not known to either the subjects or the investigator until the study is completed and the data analyzed. All subjects receive Cetaphil cleanser, moisturizer and sunscreen for daily use throughout the study. The first application of Tube A and B is done in the presence of the study coordinator to ensure proper application. Written directions are given to the subjects to apply the products at the same time of day every day and to apply Tube B five minutes after applying Tube A.

Study participants apply the study creams once daily for 12 weeks. Clinical photographs are taken at weeks 2, 4, 8, and 12. In addition, subjects complete self assessments at each study visit. These assessments are validated by the primary investigator using a 5 point scale as compared to baseline photos: (5) much worse; (4) worse; (3) unchanged; (2) improved and (1) much improved. Subjects also assess the appearance of their arms using the same 5-point scale.

Skin firmness (elasticity) is assessed using a ballistometer. Skin retention of water/skin moisture content is assessed via impedance measurements on the cheek area of subjects using a Novameter. Transepidermal water loss is measured using a Tewameter. Sebum secretion is measured using a Sebometer and Sebutape.

Clinical assessments by both the investigator and the subject show subjective improvements of fine lines, skin thickness, dyschromia in subjects treated with either TLR-7 agonists or retinoids. This improvement is, however, greatest in subjects assigned to a regimen of both the TLR-7 agonist and tretinoin. Sebometer measurements are unchanged in all groups except those treated with the TLR-7 agonist alone (increased sebum production). Tewameter measurements show increased TEWL in only the subjects treated with tretinoin alone. TEWL is observed as unchanged or decreased in those treated with the TLR-7 agonist (IRM) alone. TEWL is observed to decrease from baseline in almost all subjects treated with TLR-7 and tretinoin, but this is seen only at the 12-week study visit. Irritation (erythema and scaling) is seen in subjects treated with retinoid alone at all time points but maximally at the 2 and 4 week time points. Subjects treated with the combination of retinoid and TLR-7 agonist show mild irritation at weeks 2 and 4, but none at week 8.

2b—Combined Day/Night Treatment of Aging Skin with IRM and Retinoid

The above study is repeated except that the topical composition containing the IRM is applied in the morning and a topical composition containing tretinoin is applied at night.

After completion of the study, based on SIAscopy, participants exhibiting pronounced SFLs show a statistically significant increase of collagen above baseline. This is also evidenced in subjective assessment of an improved appearance of the skin, and measurements showing increased elasticity.

Tewameter measurements show increased TEWL in only the subjects treated with tretinoin alone. TEWL is observed to be unchanged or decreased in subjects treated with the TLR-7 agonist alone. TEWL is observed to decrease from baseline in almost all subjects treated with TLR-7 and tretinoin, at the 8 and 12 week time point. Irritation (erythema and scaling) is seen in subjects treated with the retinoid alone at all time points but maximally at the 2 and 4 week time points. Subjects treated with both retinoid and TLR-7 agonist are observed with mild scaling at the 2-week time point, but no erythema at any time points.

Standard 2-4 mm skin punch biopsies are taken at pre-identified sites (on subject's arms) on Day 0 and at the Week 12 visit. Subjects apply the assigned formulation in the presence of the investigator and return for a biopsy within 4-6 hours.

Histopathological changes are observed after completion of the 12-week trial. In the epidermis, one or more of the following changes are noted in the skin at non-placebo study sites (i.e., site of application of study composition): compact stratum corneum; ordered epidermal proliferation; a more regular rete ridge pattern (less flattening).

In the dermis, one or more of the following changes are noted in the skin at non-placebo study sites: increase in papillary dermal hyperplasia; increased dermal thickness; reduction in solar elastosis; a more uniform pattern of stromal cells within the papillary dermis; an increase of stromal cells within the area of solar elastosis; increased collagen staining; and/or increased hyaluronic acid staining.

Combined Treatment of Aging Skin with IRM and Retinoid and Antioxidant

The same study in subsection 2.b (IRM applied in the morning in combination with retinoid applied at night) is performed in an open label fashion with the addition of application of Tube C containing an antioxidant (Argan Oil) at a time point 6 hours prior to the application of Tube A and Tube B. (All Tube A contains 5% IRM; all Tube B contains either 0.025% tretinoin.) Similar results are reported except no groups showed irritation at any time point.

Gene Expression

The effects of topical compositions containing targeted IRMs in the above-described embodiments are further investigated for their effects on gene expression. Standard 2-4 mm skin punch biopsies are taken at pre-identified sites (on subject's arms) on Day 0 and at the Week 12 visit. Subjects apply the assigned formulation in the presence of the investigator and return for a biopsy within 4-6 hours. The biopsies are placed in RNALater (Ambion, Austin, Tex.), incubated overnight at 4° C., and then stored at −70° C. until processing. Total RNA is extracted, purified and quantified using the Qiagen RNeasy Mini Kit (Qiagen, Inc, Valencia, Calif.). Purified RNA is reverse transcribed using the SuperScript Single Stranded cDNA Synthesis Kit (Invitrogen Corp, Gaithersburg, Mass.). Real-time reverse transcriptase polymerase chain reaction is performed using TaqMan arrays (Applied Biosystems, Foster City, Calif.). 96 well arrays, containing reagents for 23 or 47 genes, and a control (glyceraldehyde 3-phosphate dehydrogenase (GAPDH) are used.

PCR is performed using 1 ng cDNA for 35 cycles of 30 seconds at 97° C. and 1 minute at 59.7° C., preceded by incubation for 2 minutes at 50° C. and 10 minutes at 94.5° C. using an ABI PRISM 7900HT Sequence Detection System (Applied Biosystems). Amplification cycle threshold (CT) values are normalized for each subject using the average GAPDH value for that subject only. For each gene examined, at test is performed on replicate normalized CT values for each pairing of IRM-containing formulation and its placebo. Pairings with statistically significant differences at the 95% confidence level are used to calculate the fold change in expression.

The efficacy of the IRMs in the above-described embodiments is further demonstrated in the following non-limiting in vitro experiments.

Cultured human epidermal keratinocytes are grown to confluency in a 6-well plate under appropriate culture conditions. At confluency, three of the six wells are treated with culture media supplemented with test material (IRM) at concentrations of 0.01%. The remaining three wells are treated with culture media alone and act as a control. After applying the test material, the cells are incubated for 24 hours at 37±2° C. and 5±1% CO₂. At the end of the incubation period, the culture media is removed via aspiration and the cells are washed once with cold phosphate buffer solution (PNS) using approximately 1 ml per well. After the wash, a trypsin/EDTA solution is added to the wells to detach the cells. Trypsin neutralizing solution is then added to the wells.

The treated cells and the untreated cells are pooled into respective 15 ml centrifuge tubes and pelleted by centrifuging at 1000 RPM at 4±2° C. After removing the supernatant, the pelleted cells are lysed by adding 900 μl of guanidinium thiocyanate lysis solution to each tube and then repeatedly drawing and releasing the solution into the pipette until the cell pellet is dissolved. The cell lysates are stored at −75° C. until the RNA extraction process as described below is completed.

RNA Isolation:

RNA isolation is performed using the RNAqueous Kit from Ambion Inc. (Austin, Tex.). To the cell lysates or tissue homogenates prepared above, an equal volume of 64% ethanol is added and the tubes are vortexed. Up to 700 μl of the mixture is transferred to a glass fiber filter cartridge, which is loaded into a 1.5 ml collection tube and the cartridge is centrifuged for 1 minute at 14,000 RPM. The flow through (filtrate) is discarded. Any remaining mixture is loaded into the filter cartridge and the centrifugation process is repeated until all of the mixture has been processed.

The filter is then washed to remove any residual cellular debris from the RNA bound to the glass fibers by applying 700 μl of a first wash solution (1 time) and 500 μl of a second wash solution (2 times) to the filter cartridge and centrifuging at 14,000 RPM for 1 minute to pass each wash through the cartridge. The flow through is discarded after each wash.

After the final wash, one final spin is performed without wash solution to remove any residual wash solution in the filter cartridge. The RNA bound to the glass fibers within the cartridge is then be eluted by applying 30 μl of Tris-EDTA buffer (10 mM Tris-HCl, 1 mM EDTA, preheated to 70°-80° C., hereinbelow “TE buffer”) to the cartridge and centrifuging the cartridge over a fresh collection tube at 14,000 RPM for one minute.

For samples prepared from cell lysates and small tissues, the elution process is repeated with an additional 30 μl of preheated TE buffer. For samples prepared from larger tissues (i.e., full thickness tissues) the elution process is repeated two additional times. After the RNA is eluted, RNA concentration is quantified using a Ribogreen assay. RNA quality is assessed via gel electrophoresis.

RNA Concentration Assay:

Ribogreen reagent is provided as a stock solution in DMSO. Prior to use, the reagent is diluted 2000 fold in TE buffer. The RNA assay requires 200 μl of diluted Ribogreen reagent per sample to be tested and 1 μl of the reagent for the standards. Once prepared, the diluted reagent is stored protected from light. A series of RNA standards are prepared by diluting purified ribosomal RNA derived from E. coli to the following concentrations: 2 μg/ml, 1 μg/ml, 200 ng/ml, 40 ng/ml and 0 ng/ml (blank). Prior to assaying, the RNA samples prepared above are diluted 1000 fold in TE buffer. For the RNA assay, 100 μl of the diluted samples or standards are transferred to the wells of a black 96-well plate. The samples and standards are assayed in duplicate. After the samples/standards are added to the plate 100 μl of diluted Ribogreen assay reagent is added to the wells and the plate is gently mixed and allowed to incubate for 5-10 minutes protected from the light. After this incubation, the plate is read with a fluorometer using an excitation wavelength of 500 nm and an emission wavelength of 525 nm.

RNA Gel Electrophoresis:

An RNA gel (1%) is prepared by adding 0.3 g agarose to 21.6 ml diethylpyro-carbonate (DEPC) treated water. The agarose is dissolved by boiling the water in a microwave oven. After the solution is cooled to approximately 55° C., 5.4 ml of formaldehyde and 3.0 ml 10×MOPS (0.2 M MOPS [pH 7.0], 20 mM sodium acetate, 10 mM EDTA, made in DEPC H₂O and filter sterilized). After mixing, the agarose gel is cast in the horizontal gel apparatus with loading slots placed on the side of the gel closest to the negative terminal. The gel is allowed to set for at least 1 hour at room temperature.

While the gel is setting, 175 ml of 1×MOPS is prepared by diluting the 10× stock. After the gel is set, the comb is removed and the buffer chamber of the gel apparatus is filled with 150-175 ml 1×MOPS (enough buffer is added to cover the gel with approximately 3 mm of buffer). The cover is placed on the apparatus, the electrical leads are attached to the power source, and the empty gel is run at 40 V (4 V/cm) for 5-10 minutes.

While the gel is running, the RNA samples are prepared by transferring approximately 1 μg of each sample RNA to a 600 μl PCR tube. DEPC H₂O is used to bring the total volume of all the samples to a common level and then 1-3 volumes of a gel-loading buffer (i.e. 5% glycerol, 1 mM EDTA, 0.025% bromophenol blue, 0.025% xylene cyanol FF, 20% formaldehyde, 50% formamide, 10 μg/ml ethidium bromide) are added. The samples are denatured by placing them at 65-70° C. for 5-15 minutes and then placed on ice to cool. The samples are then carefully loaded into the lanes (each loading slot can hold 10-15 μl of sample, depending upon the thickness of the gel) and run on the gel at 40 V for 1-3 hours. At the end of the run, the RNA is visualized by placing the gel on a UV transilluminator. An RNA sample is used for subsequent processing if both the 18S and 28S ribosomal bands are clearly visible and there is little or no staining below the 18S band.

mRNA Amplification:

mRNA is amplified using the MessageAmp aRNA kit from Ambion Inc. according to the following protocol.

First Strand cDNA Synthesis:

To start the first strand synthesis, 5 μg of total RNA for each sample are added to 600 μl PCR tubes and the total volume of liquid in the tube is adjusted to 12 μl with DEPC H₂O. To each tube, 1 μl of T7 Oligo(dT) primer is added and the tube is incubated at 70±2° C. for 10 minutes to denature the RNA and is then placed on ice to allow the primer to anneal to the poly A ends of the mRNA. After cooling, 2 μl of 10× first strand buffer, 1 μl of RNAse inhibitor and 4 μl of dNTP mix is added to each tube, and the tube is placed at 42° C. As soon as the tube is heated, 1 μl of reverse transcriptase is added and the tubes are returned to 42±2° C. for 2 hours. At the end of the two hours, the tubes are briefly centrifuged to collect all of the fluid at the bottom of the tube and then placed on ice.

Second Strand Synthesis and cDNA Purification:

For the synthesis of the second strand of cDNA the following ingredients are added sequentially to the tubes: 63 μl DEPC H₂O, 10 μl 10× second strand buffer, 4 μl dNTP mix, 2 μl DNA Polymerase and 1 μl of RNAse H. The contents of the tube are mixed and then incubated at 16±2° C. for 2 hours.

Towards the end of the 2 hour incubation, a sufficient quantity of DEPC H₂O is warmed to 50±2° C., and a cDNA purification filter cartridge is equilibrated with 50 μl of cDNA binding buffer (one cartridge per sample) for at least 5 minutes. After incubation is complete, 250 μl of cDNA binding buffer are added to each tube and thoroughly mixed. The contents of the PCR tube are then transferred to the cDNA purification filter cartridge. The cartridge is then placed in a collection tube and centrifuged at 10,000 RPM for 1 minute. The flow-through is discarded and 650 μl of cDNA wash solution is added to the cartridge. The cartridge is centrifuged again; the flow-through is discarded, and is then centrifuged one additional time to ensure that the wash buffer has been completely emptied from the filter. The cDNA is eluted by applying 10 μl of preheated DEPC H₂O to the filter and centrifuging the filter in a new collection tube at 10,000 RPM for one minute. This elution is performed one additional time to give a total volume of 16-18 μl of cDNA solution.

In Vitro Transcription to Synthesize aRNA and aRNA Purification:

The in vitro transcription begins by adding the following to the cDNA solution: 4 μl each of T7 ATP solution, T7 CTP solution, T7 GTP solution, T7 UTP solution, 4 μl of 10× Reaction buffer, and 4 μl of T7 enzyme mix. The contents of the tube are mixed and the tube and contents are incubated at 37±2° C. for 6-14 hours.

Towards the end of the incubation, a sufficient volume of Elution Solution is warmed to 50-60° C. and an aRNA filter cartridge is equilibrated with 100 μl of aRNA binding buffer for at least 5 minutes. At the end of the incubation period, 350 μl of aRNA binding buffer are added to the sample tubes and thoroughly mixed. An additional 250 μl of absolute ethanol are also added to each tube. The mixture is then transferred to an aRNA filter cartridge; the cartridge is then inserted into a collection tube and centrifuged at 10,000 RPM for 1 minute. The flow-through is discarded and 650 μl of aRNA wash buffer is added to the cartridge followed by centrifuging at 10,000 RPM for one minute. After discarding the flow-through, the cartridge is spun one final time to remove all traces of the wash buffer. The cartridge is then transferred to a new collection tube and 25 μl of pre-warmed Elution Solution is added to the cartridge. The cartridge is incubated for 2 minutes at room temperature and then aRNA is eluted by centrifuging for 1 minute at 10,000 RPM. This elution is performed one additional time to give a total volume of 45-50 μl of aRNA solution. The final concentration of the aRNA is determined by the Ribogreen assay described above. In addition, the quality of the aRNA is checked via gel electrophoresis as described above. An aRNA sample is used for subsequent processing if a broad band of RNA is observed.

Labeling and Purification of aRNA:

aRNA is labeled with fluorescent dyes using the PerkinElmer ASAP RNA Labeling Kit. Two tubes are prepared for the labeling process—for the untreated sample Cy3 labeling (green), and for the treated sample Cy5 labeling (red). To the Cy3 tube are added 2 μg of aRNA prepared from the untreated/control sample and enough DEPC H₂O to bring the total volume up to 4 μl is added.

To the Cy5 tube are added 2 μg of aRNA prepared from the sample treated with the test material and enough DEPC H₂O to bring the total volume up to 4 μl is added. To both tubes, 5 μl of ASAP labeling buffer and 1 μl of the specific dye for the tube (Cy3 or Cy5) are added. The tubes and contents are incubated for 15 minutes at 85±2° C. At the end of the 15 minutes, the tubes are placed on ice to cool and then 2.5 μl of ASAP stop solution are added to each tube. The above proportions are sufficient for analyzing one microarray chip. If more chips are to be used then the labeling is increased proportionately.

To purify the labeled aRNA, a microcon YM-30 filter column is inserted into a collection tube and filled with 400 μl of TE buffer. The Cy3 and Cy5 probes are combined (12.5 μl of each) and then added to the microcon filter and thoroughly mixed with the TE buffer. The filter is centrifuged at 12,000 RPM for 8 minutes and the flow through is discarded. The column is washed twice with 400 μl of TE buffer, discarding the flow though each time. After the final wash, the filter column is inverted, placed into a new collection tube and centrifuged at 12,000 RPM for 2 minutes to collect the probe (the probe is concentrated in a volume of 2-30 μl of residual TE buffer).

Microarray Hybridization and Washing:

For hybridization, 45 μl of 10× control target RNA (supplied with Agilent Technologies In Situ Hybridization Kit) is mixed with 160 μl of DEPC water and 9 μl of 25× Agilent Fragmentation Buffer. This mixture is incubated at 60° C. for approximately 30 minutes in a hybridization oven. At the end of the incubation, 225 μl of Agilent Hybridization Buffer is added along with the fluorescent aRNA probes prepared above. The mixture is then incubated at 70° C. for 5-10 minutes in a water bath. During this incubation period, an Agilent SUREHYB hybridization chamber is prepared by inserting a glass gasket slide into the bottom half of the chamber. At then end of the incubation, the hybridization mixture (approximately 450 μl) is applied to the glass gasket slide and an Agilent Human 1A Oligo Microarray Chip is placed face down on top of the gasket such that the hybridization solution is sandwiched between the glass gasket slide and the microarray face of the chip. The top half of the chamber is attached and the connecting thumbscrew tightened. After verifying that there is good bubble formation in the chamber, it is placed into the hybridization oven for approximately 17 hours (at 60° C., rotating at 4 RPM). At the end of the hybridization period, the microarray/glass gasket is removed from the SUREHYB chamber and placed in 50 μl of a first wash solution (room temperature, 6×SSC, 0.005% Triton X-102) to release the gasket.

After the gasket has fallen away from the microarray, the array is transferred to 300 μl of fresh wash solution 1 on a magnetic stir plate. The array is washed while the solution is mixed at medium speed for 10 minutes and is then transferred to 300 μl of wash solution 2 (0.1×SSX, 0.005% Triton X-102, 4° C.) for 5 minutes. After the final wash, the array is centrifuged at 500 RPM (for approximately 5 minutes) until dry.

Microarray Scanning and Analysis:

The microarrays are scanned with an Axon GenePix 4100A Scanner with the scanning resolution set to 10 μm and analyzed with GenePix Pro software. During the initial scan the PMT gains for the scanner are adjusted such that the Cy5/Cy3 image count ratios are between 0.88 and 1.12. A standard curve for the Ribogreen assay is prepared by plotting the relative fluorescent units versus the known RNA concentrations in μg/ml for the standards and further by regression analysis to establish the line that best fits these data points. Mean RFU values for the test materials and untreated samples are then used to estimate the amount of RNA present in each sample. The level of gene expression is related to the fluorescence intensity of the probed gene marker on the microarray. Fluorescence measurements between the Cy3 and Cy5 probes are normalized. The total fluorescent signal for both dyes is normalized with a correction factor such that the ratio of total intensities for both dyes equal to one.

Criteria for evaluating changes in gene expression are known to those of ordinary skill in the art and include the following: First, the ratio of Cy3/Cy5 (untreated/treated) fluorescence intensity is greater than 1.5 or less than 0.66. (This relates to a change in gene expression of at least +/−30%.) Second, the fluorescence intensity of the gene marker is greater than the background intensity. Third, the gene feature is clearly marked specifically by the aRNA probes and is not due to non-specific fluorescence. The first two criteria are filtered via computer analysis. The last criterion requires visual inspection of the array.

Ratios of greater than about 1.3 are interpreted to indicate that a gene is upregulated by the treatment, whereas ratios of less than about 0.7 are interpreted to indicate a down-regulated gene. Accordingly, a ratio of 1.3, where the treated value is 130% of the untreated value, indicates a 30% increase in gene expression. Similarly, a ratio of 0.7 means that the treated value was 70% of the untreated value, indicating a 30% decrease in gene expression.

In both the biopsy experiments and the in vitro experiments, the expression of the following genes are observed as upregulated: COL1; COL3; TIMP1. Decreased expression of Matrix Metalloproteinases 1, 2 and/or 9 is also observed. The following are also observed in biopsies of some study participants or in vitro testing: increases the expression of one or more of (a) Microfibril-Associated Glycoprotein-1 (MAGP-1), (b) Fibrillin-1 and/or (c) Fibulin 5 (FBLN5); decreases in the expression of one or more of (d) Latent-Transforming Growth Factor Beta-Binding Protein 2 (LTBP)-2, (e) Sirtuins 1 and/or 4 (SIRT1, SIRT4) and/or (f) Lysyl Oxidase-Like Enzyme (LOXL1).

### Examples

### In Vitro Testing Show Changes in Cytokine Production

Cytokine production by plasmacytoid dendritic cells is measured using real-time RT-PCR on RNA collected from peripheral blood mononuclear cells (PBMC) including pDC (plasmacytoid dendritic cells). In these assays, pDCs produce TNF-α, IL-8, IFN-α, IL-12p40, IL-1RA, IL-6, IP-10, MCP-1, MIP-1a, MIP-3a, and TNF-a, in addition to type 1 IFNs.

An IRM (TLR-7 agonist) as described in one of the four embodiments of the present invention is introduced into a 24-well plate containing PBMC and pDC h Control wells are treated with DMSO. Cells are incubated for either one or two hours at 37° C. At the indicated times the cells are harvested by gently pipeting the cells into a 1.5 mL Eppendorf tube and centrifuging at 400 RCF for 10 min at 4° C. The supernatant is removed from the tube and the cells are lysed with 1 mL TRIzol (Life Technologies). RNA is purified from the samples and treated with DNase I (Life Technologies) to remove genomic DNA, after which the samples are re-extracted with TRIzol. Final pellets are suspended in and diluted 1:100 in water. RNA is quantified by absorbency (Abs260) and is reverse-transcribed using SuperScript First Strand Synthesis System for RT-PCR (Life Technologies). Primers for quantitative PCR are generated using Primer Express (PE Applied Biosystems). Each primer set is designed to amplify genomic DNA and is tested against a sample of human genomic DNA to verify the amplicon size. Quantitative PCR is performed on an ABI PRISM 7700 Sequence Detector. Amplified products are detected using SYBR Green PCR Master Mix (PE Biosystems). PCR is performed for 35 cycles for 15 seconds at 95° C. and 1 min at 60° C., preceded by a 2 minute incubation at 50° C. and 10 minute incubation at 95° C. The instrument software calculates the number of cycles, designated Ct, required for the accumulated signal to reach a designated threshold value at least 10 standard deviations greater than the baseline. The Ct value is proportional to the number of starting copies of the target sequence. Relative quantitation of gene expression was performed using the DDCt method (User Bulletin #2, PE Applied Systems).

Cytokine and chemokine protein levels are measured from tissue culture supernatants or cell extracts using commercially-available enzyme-linked immuno assay (ELISA) kits. Human TNF, IL-12, IL-10 (standard IL-10 assay and IL-10 Ultrasensitive), IL-6, IL-1RA, MCP-1, and Mip-1a ELISA kits are available, for example, from BioSource. Human Mip-3a ELISAkit is available from R&D Systems. Multi-species IFN-a and IFN-x ELISA kits are available from PBL Biomedical Laboratories. Human IP-10 ELISA kits are available from Cell Sciences (Norwood, Mass.). The multi-species IFN-a ELISA assay specifically detects all of the human IFN-a subtypes, except IFNA21. Results show that plasmacytoid DC produces TNF-α, IL-8, and other cytokines, in addition to IFN-α. TNF-a production from the same cells induce PBMC cultures to secrete IL-12p40, IL-1RA, IL-6, IP-10, MCP-1, MIP-1a, MIP-3a, and TNF-α, in addition to type 1 IFNs. The IRM (TLR-7 agonist) treated pDC cultures produce 2-20 times more IFN-α than untreated pDC cells. Interferon gamma is also observed to increase in cells treated with the TLR-7 agonist according to methods of the present invention.

The above studies are replicated with all trans-retinoic acid being added at concentrations ranging from 0.02% to 1% to the culture wells at the same time as TLR-7 agonists. Results show that all trans-retinoic acid lowers the amount of IFN-γ that is expressed by the PBMC cells upon exposure to the TLR-7 agonist.

### Formulation Example

The following is an illustrative examples of a topical formulation that can be used in the practicing the inventive methods the present invention. The components and specific ingredients are presented as being typical, and various modifications can be derived in view of the foregoing disclosure within the scope of the invention.

| Phase | INCI Name | % (wt/wt) |
|---|---|---|
| A | Carbomer | 0.5 |
| A | Water | 90.5 |
| B | Immune Response Modifier (IRM)* | 5.0 |
| C | Helianthus Annus (Sunflower) Seed Extract | 1.0 |
| C | Panthenol | 1.0 |
| C | Aloe Vera | 1.0 |
| D | Phenoxyethanol (and) Methylparaben (and) | 1.0 |
| | Ethylparaben (and) Butylparaben (and) Propylparaben | |
| | (and) Isobutylparaben | |

*IRM may be selected from first, second, third, fourth or fifth embodiments.

Combine Phase A ingredients and mix until uniform at room temperature. Add Phase B to Phase A with mixing. Add Phase C ingredients to Phase A/B while mixing. Add Phase D to Phase A/B/C and mix well.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

### APPENDIX A

### Baumann Facial Photoaging Visual Scale

Evaluations are based at looking at half—not full face.

Periorbital—Entire area; not limited to lines at the lateral canthus but all lines in this area.

1=0-1 Short Fine Lines (less than 1 cm)

2=2-3 Short Fine Lines (less than 1 cm)

3=4+ short fine lines or 1-3 long fine lines (longer than 1 cm)

4=4-5 long fine lines (longer than 1 cm) or 1 line of moderate depth.

5=2 lines of moderate depth or at least 6 long fine lines (longer than 1 cm).

6=2 or more lines of moderate depth and at least one of them is longer than 1 cm. No deep lines

7=At least 1 deep line or 5 moderately deep lines longer than 1 cm

8=>5 moderately deep lines longer than 1 cm, or 2 deep lines, or 1 deep line longer than 1 cm.

9=Redundancy (folding) of at least 1 lines, or 3 or more deep lines, or 2 deep lines longer than 1 cm

Perioral—Limited to the vertical lines along the border of the upper and lower lip

1=None

2=1-2 short fine lines (Less than 1 cm)

3=3 short fine lines, or 2 long fine lines (greater than 1 cm long) No lines cross the vermillion border

4=1-2 moderate deep lines or 3 long fine lines (greater than 1 cm) or 1-2 fine lines that cross the vermillion border.

5=1 moderately deep line (at least 1 cm long) or 3-5 moderate lines or 3 fine lines that cross the vermillion border

6=6 or more moderate lines, or 2 or more moderately deep lines at least 1 cm long or 1 moderately deep line that crosses the vermillion border. No deep lines.

7=1 deep line or 2 or more moderately deep lines that cross the vermillion border.

8=2-3 deep lines, or 1-2 deep lines crossing vermillion, 1-3 deep lines that are greater than 1 cm.

9=4 or more deep lines crossing vermillion or 4 or more deep lines longer than 1 cm\

Dyschromia

1=0-2 macules

2=3-10 light brown macules

3=10 or more light brown macules or 1 light brown patch

4=11 or more light brown macules, or 1 moderately dark patch, or 1-4 moderately dark macules

5=25% involvement of face with light brown macules and patches, or more than 4 moderate brown macules.

6=25%-50% of face with light brown macules or patches, or 1-3 moderate (medium brown) patches.

7=4+ or more moderate (medium) brown patches, or 25%-49% of face with medium brown macules and patches. 1-5 Dark brown/black macules. No dark brown/black patches.

8=25% of face with dark brown patch or dark black patch, or greater than 5 dark macules

9=More than 50% of face dark brown/black macules and patches.

Diffuse Redness

1=0% of face, no pink

2=Light pink or 10% of face

3=Light pink or 25% of face

4=Medium pink on < or =25% of face

5=Medium pink or 26-50% of face

6=Medium pink or >50% of face

7=Dark red on 10% of face

8=Dark red on 26-50% of face

9=Dark red on >50% of face.

What is claimed is:

1. A method of reducing the appearance of skin aging comprising the step of applying a topical composition to the skin of a human exhibiting one or more signs of skin aging selected from fine lines, wrinkles, decreased skin elasticity, decreased light reflection, uneven pigmentation (dyschromia), coarse texture, dryness, solar lentigos, seborrheic keratoses, decreased dermal thickness, and/or increased sebum production wherein the composition consists essentially of a dermatologically acceptable carrier and a targeted immune response modifier selected from the group consisting of: (i) a 5-amino thiazolo[4,5-d]pyrimidin-one compound; (ii) an 8-hydroxyadenine compound; (iii) an imidazo-1,5-napthyridine compound; (iv) a 2-substituted-8-oxodeazapurine; (v) methyl 2-(3-{[6-amino-2-butoxy-8-oxo-7H-purin-9(8H)-yl]methyl}phenyl)acetate; (vi) 2-(3-{[6-amino-2-butoxy-8-oxo-7H-purin-9(8H)-yl]methyl}phenyl)acetic acid; and (iii) methyl 2-(3-(((3-(6-amino-2-butoxy-8-oxo-7H-purin-9(8H)-yl)propyl)(3-(dimethylamino)propyl)amino)phenyl)acetate and (vii) 2-(3-(((3-(6-amino-2-butoxy-8-oxo-7H-purin-9(8H)-yl)propyl)(3-(dimethylamino)propyl)amino)phenyl)acetic acid.

2. The method of claim 1 comprising the further step of applying a retinoid to the skin of a human exhibiting one or more signs of skin aging selected from fine lines, wrinkles, decreased skin elasticity, decreased light reflection, uneven pigmentation (dyschromia), coarse texture, dryness, solar lentigos, seborrheic keratoses, and decreased dermal thickness, and/or altered sebum production.

3. The method of claim 1 or claim 2 comprising the further step of applying an antioxidant or an anti-inflammatory agent to the skin of a human exhibiting one or more signs of skin aging selected from fine lines, wrinkles, decreased skin elasticity, decreased light reflection, uneven pigmentation (dyschromia), coarse texture, dryness, solar lentigos, seborrheic keratoses, and decreased dermal thickness, or increased sebum production.

4. The method of any of claims 1, 2 and 3 comprising the further step of administering a dermatologic/aesthetic procedure selected from (i) Light Emitting Diode (L.E.D.) Light Therapy; (ii) Intense Pulsed Light (I.P.L.) Therapy; (iii) laser skin resurfacing; (iv) mechanical exfoliation; (v) superficial, medium depth or deep chemical peels; (vi) radiofrequency treatment; (vii) ultrasound treatment; (viii) intradermal and intraepidermal injections with hyaluronic acid and derivatives thereof; (ix) cryosurgery or (x) botulinum toxin (applied topically or injected).
